(19) United States
(12) Patent Application Publication
(10) Pub. No.: US 2020/0144502 A1
(43) Pub. Date: May 7, 2020

(54) COVER LAYER MOLECULAR STRUCTURE, PREPARATION METHOD THEREFOR, AND CORRESPONDING OLED DEVICE

(71) Applicant: Wuhan China Star Optoelectronics Semiconductor Display Technology Co., Ltd., Wuhan (CN)

(72) Inventors: Mingming CHI, Wuhan (CN); Sangyeob LEE, Wuhan (CN); Cheguang HONG, Wuhan (CN); Xu WANG, Wuhan (CN); Xianjie Li, Wuhan (CN)

(21) Appl. No.: 16/342,535

(22) PCT Filed: Sep. 25, 2018
(86) PCT No.: PCT/CN2018/107284

§ 371 Date: Apr. 17, 2019

(30) Foreign Application Priority Data
Nov. 9, 2018 (CN) 201811058526.X

(51) Int. Cl. H01L 51/00; C07C 211/61

(52) U.S. Cl. CPC H01L 51/006; H01L 51/5275; C07C 211/61

(57) ABSTRACT

The present invention provides a cover layer molecular structure. The cover layer molecular structure includes a macromolecular structure formed by bonding a first central structure to a second central structure. The first central structure is one of 9,9-dimethyl-2-bromofluorene, 2-bromo-9,9′-spirobifluorene, and 2-bromo-9,9-diphenylfluorene. The second central structure is either of 3-p-tolyl-4-m-tolyl-diphenylamine and 4,4′-bis(3,5-xylyl)-diphenylamine.

### FIELD OF INVENTION

The present invention relates to the field of fabrication of display devices, and particularly to a cover layer molecular structure, a preparation method therefor, and a corresponding OLED device

### BACKGROUND

An active-matrix organic light emitting diode (AMOLED) is a new display technology that attracts more and more attention from the industry. The major panel manufacturers in the whole world have made efforts to dominate the OLED industry. Particularly, the manufacturers in China develop their own AMOLED panel production technology with the help of the government.

When an existing AMOLED device is encapsulated with a thin film, a cover layer is used to protect the light-emitting functional layer. However, during plasma enhanced chemical vapor deposition (PECVD), the plasma bombardment in the PECVD has impact on the cover layer and may also affect the functional layer of the device. Samsung (a South Korea company) has developed an AMOLED device that includes a layer of inorganic substance (for example, LiF) deposited by evaporation between the cover layer and an encapsulation film layer of SiN to effectively prevent the cover layer or the functional layer of the device from being destroyed by plasma.

However, deposition by evaporation of an inorganic layer such as LiF causes increased costs and a large difficulty in fabrication of the AMOLED device.

Therefore, it is necessary to provide a cover layer molecular structure, a preparation method therefor, and a corresponding OLED device, to solve the problems existing in the prior art.

### SUMMARY OF DISCLOSURE

Embodiments of the present invention provide a cover layer molecular structure having lower fabrication costs and a smaller fabrication difficulty, a fabrication method, and a corresponding OLED device, to solve the technical problems of higher fabrication costs and a larger fabrication difficulty of existing OLED devices.

### Technical Solution

An embodiment of the present invention provides a cover layer molecular structure. The cover layer molecular structure includes a macromolecular structure formed by bonding a first central structure to a second central structure.

The first central structure is one of 9,9-dimethyl-2-bromofluorene, 2-bromo-9,9′-spirobifluorene, and 2-bromo-9,9-diphenylfluorene.

The second central structure is either of 3-p-tolyl-4-m-tolyl-diphenylamine and 4,4′-bis(3,5-xylyl)-diphenylamine.

In the cover layer molecular structure according to the embodiment of the present invention, the 9,9-dimethyl-2-bromofluorene has a molecular structure of:

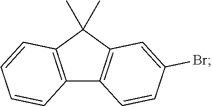 [SMILES: CC1(C)c2ccccc2-c2ccc(Br)cc21]

the 2-bromo-9,9′-spirobifluorene has a molecular structure of:

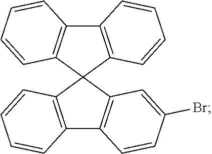 [SMILES: Brc1ccc2c(c1)C1(c3ccccc3-c3ccccc31)c1ccccc1-2]

the 2-bromo-9,9-diphenylfluorene has a molecular structure of:

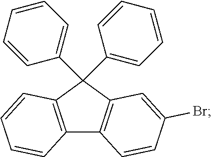 [SMILES: Brc1ccc2c(c1)C(c1ccccc1)(c1ccccc1)c1ccccc1-2]

the 3-p-tolyl-4-m-tolyl-diphenylamine has a molecular structure of:

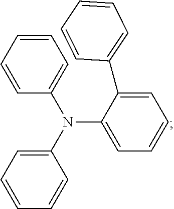 [SMILES: c1ccc(-c2ccccc2N(c2ccccc2)c2ccccc2)cc1]

and

the 4,4′-bis(3,5-xylyl)-diphenylamine has a molecular structure of:

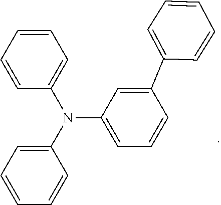 [SMILES: c1ccc(-c2cccc(N(c3ccccc3)c3ccccc3)c2)cc1]

In the cover layer molecular structure according to the embodiment of the present invention,

the macromolecular structure is:

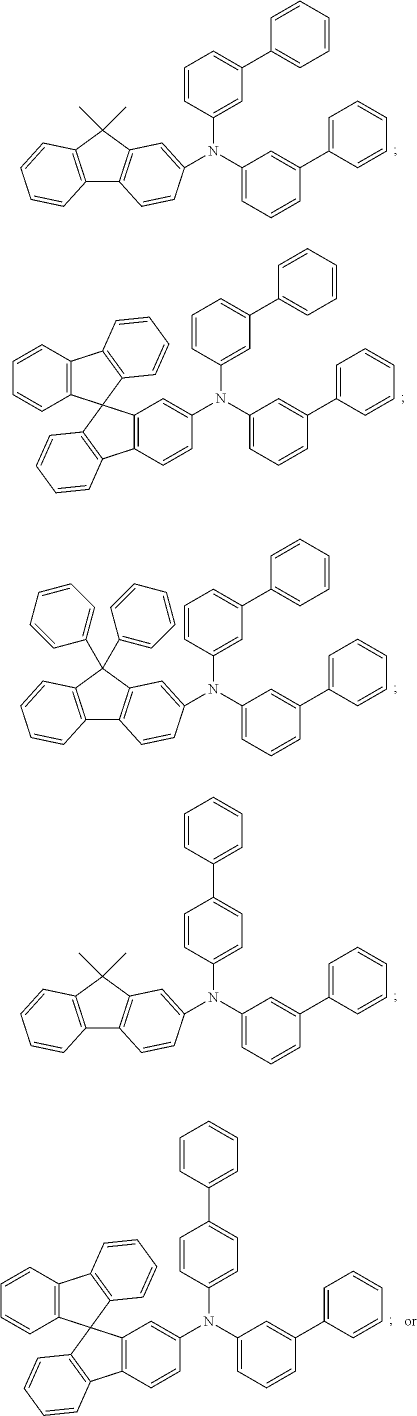 [SMILES: CC1(C)c2ccccc2-c2ccc(N(c3cccc(-c4ccccc4)c3)c3cccc(-c4ccccc4)c3)cc21 ; c1ccc(-c2cccc(N(c3cccc(-c4ccccc4)c3)c3ccc4c(c3)C3(c5ccccc5-c5ccccc53)c3ccccc3-4)c2)cc1 ; c1ccc(-c2cccc(N(c3cccc(-c4ccccc4)c3)c3ccc4c(c3)C(c3ccccc3)(c3ccccc3)c3ccccc3-4)c2)cc1 ; CC1(C)c2ccccc2-c2ccc(N(c3ccc(-c4ccccc4)cc3)c3cccc(-c4ccccc4)c3)cc21 ; c1ccc(-c2ccc(N(c3cccc(-c4ccccc4)c3)c3ccc4c(c3)C3(c5ccccc5-c5ccccc53)c3ccccc3-4)cc2)cc1]

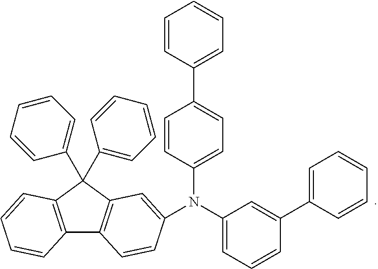 [SMILES: c1ccc(-c2ccc(N(c3cccc(-c4ccccc4)c3)c3ccc4c(c3)C(c3ccccc3)(c3ccccc3)c3ccccc3-4)cc2)cc1]

In the cover layer molecular structure according to the embodiment of the present invention, the cover layer molecular structure is produced by performing steps of:

reacting molecules of the first central structure and the second central structure for 48 hrs. in presence of palladium diacetate as a catalyst, tri-tert-butylphosphoniunm tetrafluoroborate as a ligand, and NaOt-Bu as a base in dewatered and deoxygenated toluene at 120° C., to produce the macromolecular structure.

In the cover layer molecular structure according to the embodiment of the present invention, the cover layer molecular structure has a refractivity of 1.05 to 2.15 for an incident light having a wavelength of 450 nm; and the cover layer molecular structure has a refractivity of 1.85 to 2.05 for an incident light having a wavelength of 530 nm;

In the cover layer molecular structure according to the embodiment of the present invention, the cover layer molecular structure has an extinction coefficient of 40 k to 100 k for an incident light having a wavelength of 340 to 380 nm.

An embodiment of the present invention also provides a method for preparing a cover layer molecular structure. The method includes: adding a material of a first central structure, a material of a second central structure, palladium diacetate, and tri-tert-butylphosphoniunm tetrafluoroborate to a 100 ml two-neck flask; and

under an argon atmosphere, adding dewatered and deoxygenated toluene to the two-neck flask, and reacting molecules of the first central structure and the second central structure in presence of NaOt-Bu as a base for 48 hrs. at 120° C., to obtain the cover layer molecular structure.

The first central structure is one of 9,9-dimethyl-2-bromofluorene, 2-bromo-9,9′-spirobifluorene, and 2-bromo-9,9-diphenylfluorene.

The second central structure is either of 3-p-tolyl-4-m-tolyl-diphenylamine and 4,4′-bis(3,5-xylyl)-diphenylamine.

In the method for preparing the cover layer molecular structure according to the embodiment of the present invention,

the macromolecular structure is:

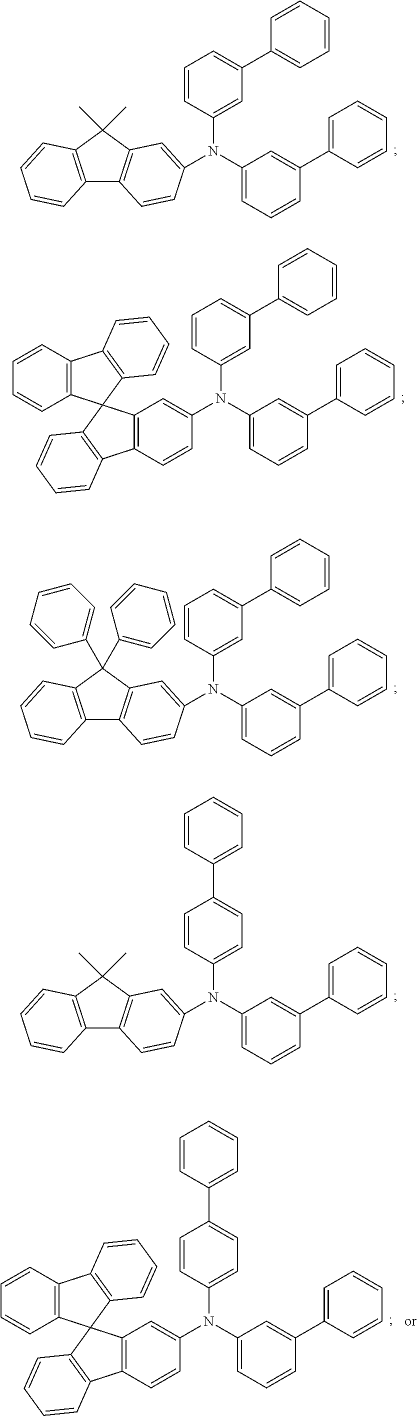 [SMILES: CC1(C)c2ccccc2-c2ccc(N(c3cccc(-c4ccccc4)c3)c3cccc(-c4ccccc4)c3)cc21 ; c1ccc(-c2cccc(N(c3cccc(-c4ccccc4)c3)c3ccc4c(c3)C3(c5ccccc5-c5ccccc53)c3ccccc3-4)c2)cc1 ; c1ccc(-c2cccc(N(c3cccc(-c4ccccc4)c3)c3ccc4c(c3)C(c3ccccc3)(c3ccccc3)c3ccccc3-4)c2)cc1 ; CC1(C)c2ccccc2-c2ccc(N(c3ccc(-c4ccccc4)cc3)c3cccc(-c4ccccc4)c3)cc21 ; c1ccc(-c2ccc(N(c3cccc(-c4ccccc4)c3)c3ccc4c(c3)C3(c5ccccc5-c5ccccc53)c3ccccc3-4)cc2)cc1]

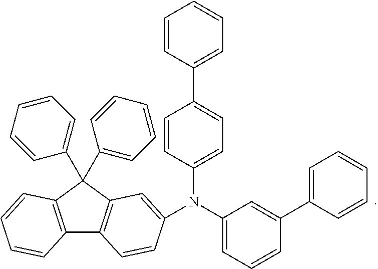 [SMILES: c1ccc(-c2ccc(N(c3cccc(-c4ccccc4)c3)c3ccc4c(c3)C(c3ccccc3)(c3ccccc3)c3ccccc3-4)cc2)cc1]

In the method for preparing the cover layer molecular structure according to the embodiment of the present invention, the cover layer molecular structure has a refractivity of 1.05 to 2.15 for an incident light having a wavelength of 450 nm;

the cover layer molecular structure has a refractivity of 1.85 to 2.05 for an incident light having a wavelength of 530 nm; and

the cover layer molecular structure has an extinction coefficient of 40 k to 100 k for an incident light having a wavelength of 340 to 380 nm.

An embodiment of the present invention further provides an OLED device. The OLED device includes an anode substrate, a hole injection layer disposed on the anode substrate, a hole transport layer disposed on the hole injection layer, a light-emitting layer disposed on the hole transport layer, a hole blocking layer disposed on the light-emitting layer, an electron transport layer disposed on the hole blocking layer, an electron injection layer disposed on the electron transport layer, a cathode substrate disposed on the electron injection layer, a cover layer disposed on the cathode substrate and an encapsulation film layer disposed on the cover layer.

The cover layer is formed by using the cover layer molecular structure according to any one of the foregoing embodiments.

### Beneficial Effects

Compared with the existing OLED device, in the cover layer molecular structure, the preparation method therefor, and the corresponding OLED device of the present invention, a cover layer molecular structure having a higher refractivity and a higher extinction coefficient is used to form a cover layer of the OLED device, so that the functional layer of the device can be effectively protected by the cover layer from being destroyed by the plasma, the fabrication costs and difficulty of the OLED device are reduced, and the technical problems of higher fabrication costs and a larger fabrication difficulty of existing OLED devices are effectively solved.

### BRIEF DESCRIPTION OF DRAWINGS

FIG. 1 is a flow chart of a method for preparing a cover layer molecular structure according to the present invention; and

FIG. 2 is a schematic structural diagram of an embodiment of an OLED device according to the present invention.

### DETAILED DESCRIPTION

The following description of various embodiments is provided to exemplify the specific embodiments of the present invention with reference to accompanying drawings. The directional terms mentioned in the present invention, for example, “upper”, “lower”, “before”, “after”, “left”, “right”, “inside”, “outside”, and “side”, are only references to the directions in the drawings. Therefore, the directional terms used are for the purpose of illustrating and understanding, instead of limiting the present invention.

In the figures, structurally similar elements are denoted by the same reference numerals.

The present invention provides a cover layer molecular structure that can be used to form a cover layer of an OLED device to protect the functional layer of the device, for example, the electron injection layer, electron transport layer, and light-emitting layer of the OLED device. In this way, the fabrication costs and difficulty of the OLED device are reduced.

The cover layer molecular structure of the present invention can be formed by bonding a first central structure to a second central structure. The first central structure is one of 9,9-dimethyl-2-bromofluorene, 2-bromo-9,9′-spirobifluorene, and 2-bromo-9,9-diphenylfluorene. The second central structure is either of 3-p-tolyl-4-m-tolyl-diphenylamine and 4,4′-bis(3,5-xylyl)-diphenylamine.

The 9,9-dimethyl-2-bromofluorene has a molecular structure of:

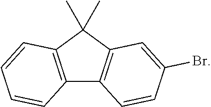 [SMILES: CC1(C)c2ccccc2-c2ccc(Br)cc21]

The 2-bromo-9,9′-spirobifluorene has a molecular structure of:

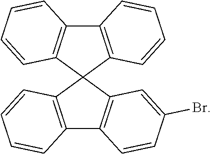 [SMILES: Brc1ccc2c(c1)C1(c3ccccc3-c3ccccc31)c1ccccc1-2]

The 2-bromo-9,9-diphenylfluorene has a molecular structure of:

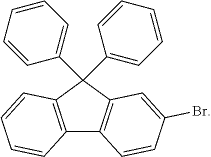 [SMILES: Brc1ccc2c(c1)C(c1ccccc1)(c1ccccc1)c1ccccc1-2]

The 3-p-tolyl-4-m-tolyl-diphenylamine has a molecular structure of:

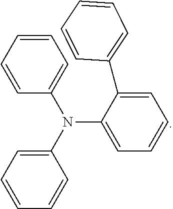 [SMILES: c1ccc(-c2ccccc2N(c2ccccc2)c2ccccc2)cc1]

The 4,4′-bis(3,5-xylyl)-diphenylamine has a molecular structure of:

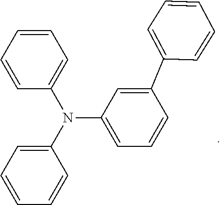 [SMILES: c1ccc(-c2cccc(N(c3ccccc3)c3ccccc3)c2)cc1]

The macromolecular structure formed by molecules of the first central structure and the second central structure is:

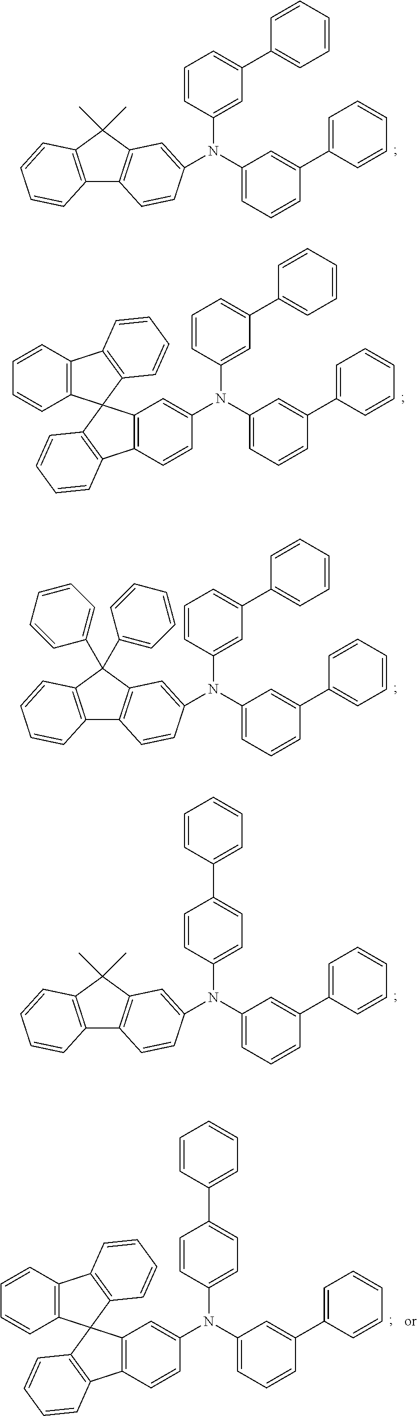 [SMILES: CC1(C)c2ccccc2-c2ccc(N(c3cccc(-c4ccccc4)c3)c3cccc(-c4ccccc4)c3)cc21 ; c1ccc(-c2cccc(N(c3cccc(-c4ccccc4)c3)c3ccc4c(c3)C3(c5ccccc5-c5ccccc53)c3ccccc3-4)c2)cc1 ; c1ccc(-c2cccc(N(c3cccc(-c4ccccc4)c3)c3ccc4c(c3)C(c3ccccc3)(c3ccccc3)c3ccccc3-4)c2)cc1 ; CC1(C)c2ccccc2-c2ccc(N(c3ccc(-c4ccccc4)cc3)c3cccc(-c4ccccc4)c3)cc21 ; c1ccc(-c2ccc(N(c3cccc(-c4ccccc4)c3)c3ccc4c(c3)C3(c5ccccc5-c5ccccc53)c3ccccc3-4)cc2)cc1]

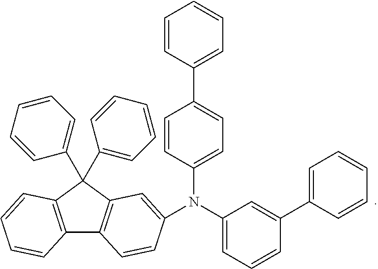 [SMILES: c1ccc(-c2ccc(N(c3cccc(-c4ccccc4)c3)c3ccc4c(c3)C(c3ccccc3)(c3ccccc3)c3ccccc3-4)cc2)cc1]

The macromolecular structure is composed of a rigid first central structure and second central structure. The first central structure and the second central structure have a certain polarity, and have a molecular weight that is not higher than 900, so that the synthesized macromolecular structure has a high refractive index. Moreover, the first central structure and the second central structure have a large conjugated structure, which ensures that the synthesized macromolecular structure has a high extinction coefficient.

Therefore, the synthesized macromolecular structure has a high glass transition temperature and a high thermal stability. That is, the material of the macromolecular structure has good low-temperature resistance, and can well stand the bombardment of high-energy particles such as plasma without degradation. The macromolecular structure has a high coefficient of absorption for light with a wavelength in the range of 250 to 400 nm.

The cover layer molecular structure according to the embodiment has a refractivity of 1.05 to 2.15 for an incident light having a wavelength of 450 nm. The cover layer molecular structure in the embodiment has a refractivity of 1.85 to 2.05 for an incident light having a wavelength of 530 nm. The cover layer molecular structure has an extinction coefficient of 40 k to 100 k for an incident light having a wavelength of 340 to 380 nm.

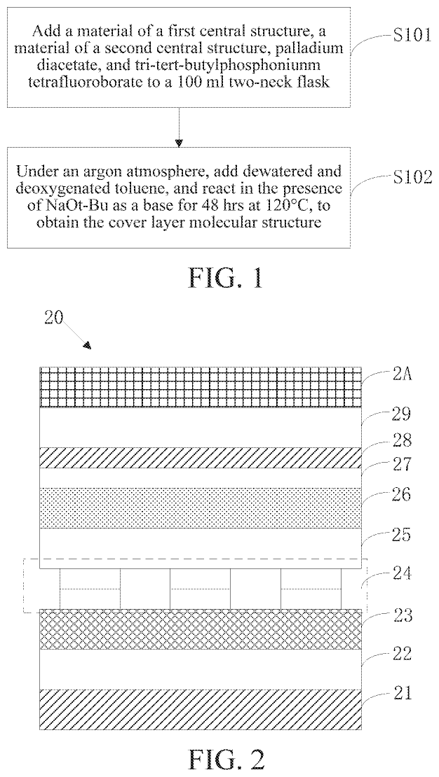

Referring to FIG. 1, FIG. 1 is a flow chart of a method for preparing a cover layer molecular structure according to the present invention. A method for preparing the cover layer molecular structure according to the embodiment includes:

Step S101: adding a material of a first central structure, a material of a second central structure, palladium diacetate, and tri-tert-butylphosphoniunm tetrafluoroborate to a 100 ml two-neck flask; and

Step S102: under an argon atmosphere, adding dewatered and deoxygenated toluene to the two-neck flask, and reacting molecules of the first central structure and the second central structure in presence of NaOt-Bu as a base for 48 hrs. at 120° C., to obtain the cover layer molecular structure.

The present invention provides an OLED device. Referring to FIG. 2, FIG. 2 is a schematic structural diagram of an embodiment of an OLED device according to the present invention. An OLED device 20 includes an anode substrate 21, a hole injection layer 22, a hole transport layer 23, a light-emitting layer 24, a hole blocking layer 25, an electron transport layer 26, an electron injection layer 27, a cathode substrate 28, a cover layer 29, and an encapsulation thin film layer 2A.

The hole injection layer 22 is disposed on the anode substrate 21, the hole transport layer 23 is disposed on the hole injection layer 22, the light-emitting layer 24 is disposed on the hole transport layer 23, the hole blocking layer 25 is disposed on the light-emitting layer 24, the electron transport layer 26 is disposed on the hole blocking layer 25, the electron injection layer 27 is disposed on the electron transport layer 26, the cathode substrate 28 is disposed on the electron injection layer 27, the cover layer 29 is disposed on the cathode substrate 28, and the encapsulation thin film layer 2A is disposed on the cover layer 29.

The cover layer molecular structure is:

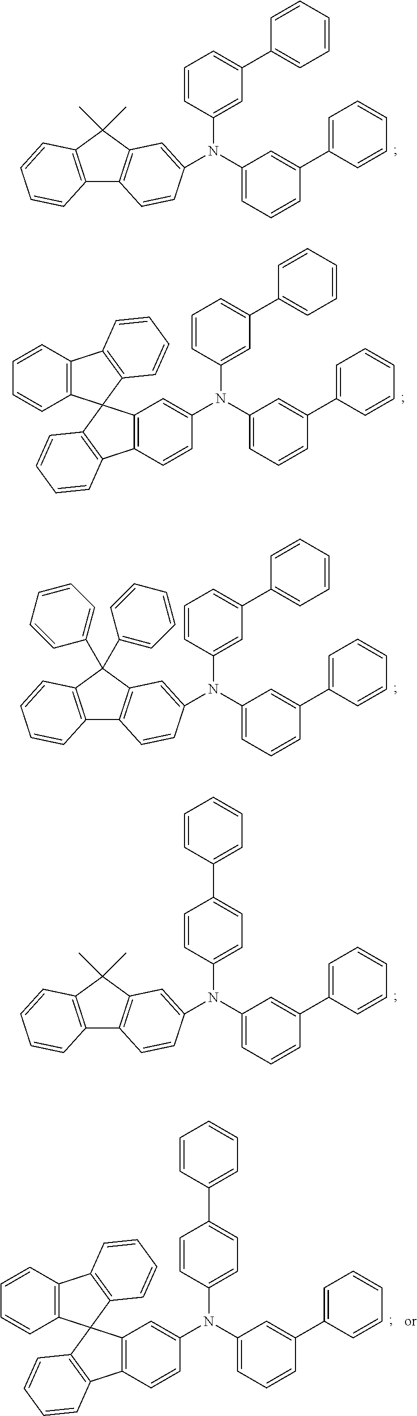 [SMILES: CC1(C)c2ccccc2-c2ccc(N(c3cccc(-c4ccccc4)c3)c3cccc(-c4ccccc4)c3)cc21 ; c1ccc(-c2cccc(N(c3cccc(-c4ccccc4)c3)c3ccc4c(c3)C3(c5ccccc5-c5ccccc53)c3ccccc3-4)c2)cc1 ; c1ccc(-c2cccc(N(c3cccc(-c4ccccc4)c3)c3ccc4c(c3)C(c3ccccc3)(c3ccccc3)c3ccccc3-4)c2)cc1 ; CC1(C)c2ccccc2-c2ccc(N(c3ccc(-c4ccccc4)cc3)c3cccc(-c4ccccc4)c3)cc21 ; c1ccc(-c2ccc(N(c3cccc(-c4ccccc4)c3)c3ccc4c(c3)C3(c5ccccc5-c5ccccc53)c3ccccc3-4)cc2)cc1]

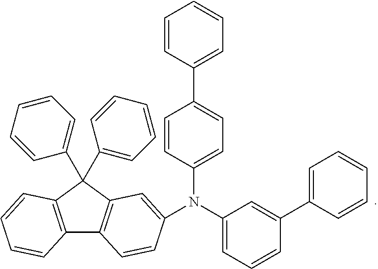 [SMILES: c1ccc(-c2ccc(N(c3cccc(-c4ccccc4)c3)c3ccc4c(c3)C(c3ccccc3)(c3ccccc3)c3ccccc3-4)cc2)cc1]

Specifically, the cover layer molecular structure is formed by bonding a first central structure to a second central structure. The first central structure is one of 9,9-dimethyl-2-bromofluorene, 2-bromo-9,9′-spirobifluorene, and 2-bromo-9,9-diphenylfluorene. The second central structure is either of 3-p-tolyl-4-m-tolyl-diphenylamine and 4,4′-bis(3,5-xylyl)-diphenylamine.

The cover layer molecular structure has a refractivity of 1.05 to 2.15 for an incident light having a wavelength of 450 nm; the cover layer molecular structure has a refractivity of 1.85 to 2.05 for an incident light having a wavelength of 530 nm; and the cover layer molecular structure has an extinction coefficient of 40 k to 100 k for an incident light having a wavelength of 340 to 380 nm.

The cover layer 29 in the OLED device according to this embodiment has a higher refractivity and a higher extinction coefficient, and no layer of inorganic substance needs to be disposed between the encapsulation thin film layer 2A and the cover layer 29 to provide protection against damage from the plasma. Therefore, the OLED device has lower fabrication costs and a smaller difficulty.

In the cover layer molecular structure, the preparation method therefor, and the corresponding OLED device of the present invention, a cover layer molecular structure having a higher refractivity and a higher extinction coefficient is used to form a cover layer of the OLED device, so that the functional layer of the device can be effectively protected by the cover layer from being destroyed by the plasma, the fabrication costs and difficulty of the OLED device are reduced, and the technical problems of higher fabrication costs and a larger fabrication difficulty of existing OLED devices are effectively solved.

In conclusion, although the present invention has been disclosed above by using preferred embodiments, the preferred embodiments are not intended to limit the present disclosure, and a person of ordinary skill in the art can make various variations and modifications without departing from the spirit and scope of the present invention. Therefore, the protection scope of the present invention should be subject to the scope defined by the claims.

What is claimed is:

1. A cover layer molecular structure, comprising a macromolecular structure formed by bonding a first central structure to a second central structure, wherein
the first central structure is one of 9,9-dimethyl-2-bromofluorene, 2-bromo-9,9′-spirobifluorene, and 2-bromo-9,9-diphenylfluorene; and
the second central structure is either of 3-p-tolyl-4-m-tolyl-diphenylamine and 4,4′-bis(3,5-xylyl)-diphenylamine.

2. The cover layer molecular structure according to claim 1, wherein the 9,9-dimethyl-2-bromofluorene has a molecular structure of: 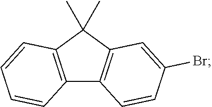 [SMILES: CC1(C)c2ccccc2-c2ccc(Br)cc21]
the 2-bromo-9,9′-spirobifluorene has a molecular structure of: 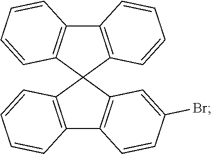 [SMILES: Brc1ccc2c(c1)C1(c3ccccc3-c3ccccc31)c1ccccc1-2]
the 2-bromo-9,9-diphenylfluorene has a molecular structure of: 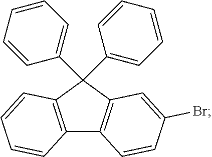 [SMILES: Brc1ccc2c(c1)C(c1ccccc1)(c1ccccc1)c1ccccc1-2]
the 3-p-tolyl-4-m-tolyl-diphenylamine has a molecular structure of: 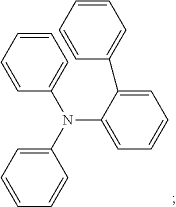 [SMILES: c1ccc(-c2ccccc2N(c2ccccc2)c2ccccc2)cc1]
and
the 4,4′-bis(3,5-xylyl)-diphenylamine has a molecular structure of:

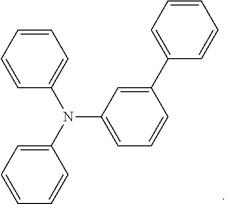 [SMILES: c1ccc(-c2cccc(N(c3ccccc3)c3ccccc3)c2)cc1]

3. The cover layer molecular structure according to claim 1, wherein the macromolecular structure is:

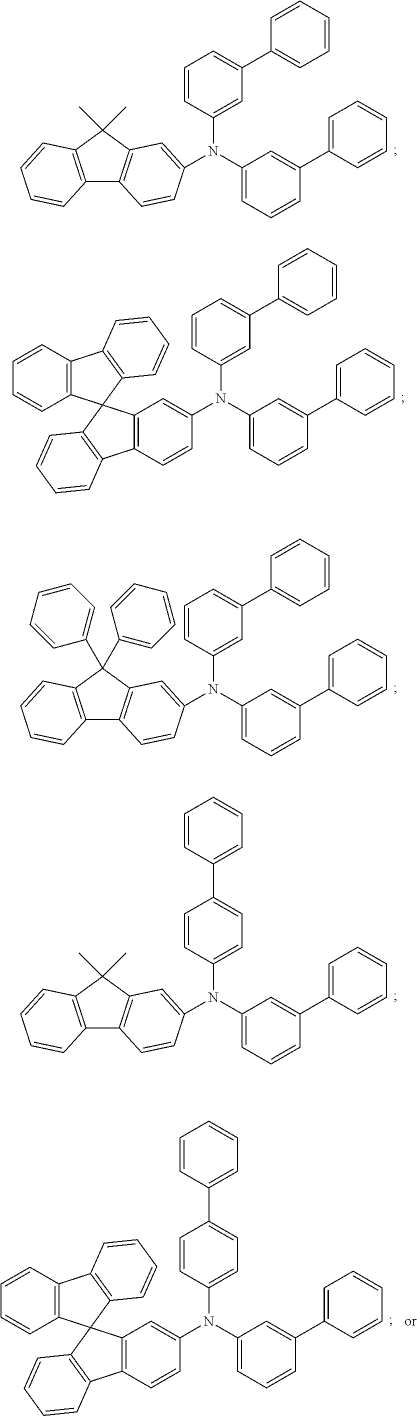 [SMILES: CC1(C)c2ccccc2-c2ccc(N(c3cccc(-c4ccccc4)c3)c3cccc(-c4ccccc4)c3)cc21 ; c1ccc(-c2cccc(N(c3cccc(-c4ccccc4)c3)c3ccc4c(c3)C3(c5ccccc5-c5ccccc53)c3ccccc3-4)c2)cc1 ; c1ccc(-c2cccc(N(c3cccc(-c4ccccc4)c3)c3ccc4c(c3)C(c3ccccc3)(c3ccccc3)c3ccccc3-4)c2)cc1 ; CC1(C)c2ccccc2-c2ccc(N(c3ccc(-c4ccccc4)cc3)c3cccc(-c4ccccc4)c3)cc21 ; c1ccc(-c2ccc(N(c3cccc(-c4ccccc4)c3)c3ccc4c(c3)C3(c5ccccc5-c5ccccc53)c3ccccc3-4)cc2)cc1]

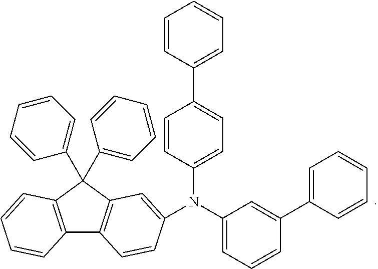 [SMILES: c1ccc(-c2ccc(N(c3cccc(-c4ccccc4)c3)c3ccc4c(c3)C(c3ccccc3)(c3ccccc3)c3ccccc3-4)cc2)cc1]

4. The cover layer molecular structure according to claim 1, wherein the cover layer molecular structure is prepared by performing steps of:
reacting molecules of the first central structure and the second central structure for 48 hrs. in presence of palladium diacetate as a catalyst, tri-tert-butylphosphoniunm tetrafluoroborate as a ligand, and NaOt-Bu as a base in dewatered and deoxygenated toluene at 120° C., to produce the macromolecular structure.

5. The cover layer molecular structure according to claim 1, wherein
the cover layer molecular structure has a refractivity of 1.05 to 2.15 for an incident light having a wavelength of 450 nm; and
the cover layer molecular structure has a refractivity of 1.85 to 2.05 for an incident light having a wavelength of 530 nm.

6. The cover layer molecular structure according to claim 1, wherein the cover layer molecular structure has an extinction coefficient of 40 k to 100 k for an incident light having a wavelength of 340 to 380 nm.

7. A method for preparing a cover layer molecular structure, comprising:
adding a material of a first central structure, a material of a second central structure, palladium diacetate, and tri-tert-butylphosphoniunm tetrafluoroborate to a 100 ml two-neck flask; and
under an argon atmosphere, adding dewatered and deoxygenated toluene to the two-neck flask, and reacting molecules of the first central structure and the second central structure in presence of NaOt-Bu as a base for 48 hrs. at 120° C., to obtain the cover layer molecular structure, wherein
the first central structure is one of 9,9-dimethyl-2-bromofluorene, 2-bromo-9,9′-spirobifluorene, and 2-bromo-9,9-diphenylfluorene; and
the second central structure is either of 3-p-tolyl-4-m-tolyl-diphenylamine and 4,4′-bis(3,5-xylyl)-diphenylamine.

8. The method for preparing the cover layer molecular structure according to claim 7, wherein
the 9,9-dimethyl-2-bromofluorene has a molecular structure of: 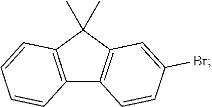 [SMILES: CC1(C)c2ccccc2-c2ccc(Br)cc21]
the 2-bromo-9,9′-spirobifluorene has a molecular structure of: 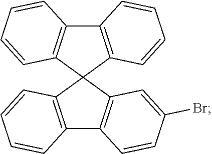 [SMILES: Brc1ccc2c(c1)C1(c3ccccc3-c3ccccc31)c1ccccc1-2]
the 2-bromo-9,9-diphenylfluorene has a molecular structure of: 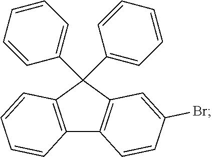 [SMILES: Brc1ccc2c(c1)C(c1ccccc1)(c1ccccc1)c1ccccc1-2]
the 3-p-tolyl-4-m-tolyl-diphenylamine has a molecular structure of: 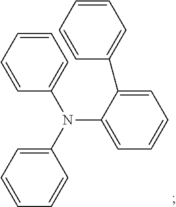 [SMILES: c1ccc(-c2ccccc2N(c2ccccc2)c2ccccc2)cc1]
and
the 4,4′-bis(3,5-xylyl)-diphenylamine has a molecular structure of:

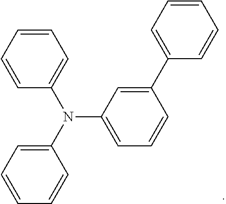 [SMILES: c1ccc(-c2cccc(N(c3ccccc3)c3ccccc3)c2)cc1]

9. The method for preparing the cover layer molecular structure according to claim 7, wherein the macromolecular structure is:

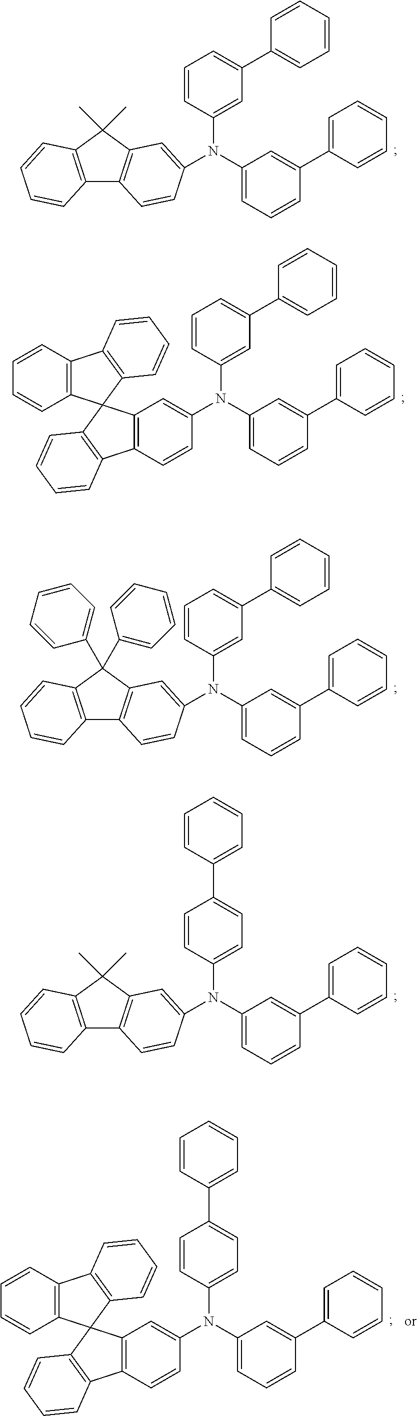 [SMILES: CC1(C)c2ccccc2-c2ccc(N(c3cccc(-c4ccccc4)c3)c3cccc(-c4ccccc4)c3)cc21 ; c1ccc(-c2cccc(N(c3cccc(-c4ccccc4)c3)c3ccc4c(c3)C3(c5ccccc5-c5ccccc53)c3ccccc3-4)c2)cc1 ; c1ccc(-c2cccc(N(c3cccc(-c4ccccc4)c3)c3ccc4c(c3)C(c3ccccc3)(c3ccccc3)c3ccccc3-4)c2)cc1 ; CC1(C)c2ccccc2-c2ccc(N(c3ccc(-c4ccccc4)cc3)c3cccc(-c4ccccc4)c3)cc21 ; c1ccc(-c2ccc(N(c3cccc(-c4ccccc4)c3)c3ccc4c(c3)C3(c5ccccc5-c5ccccc53)c3ccccc3-4)cc2)cc1]

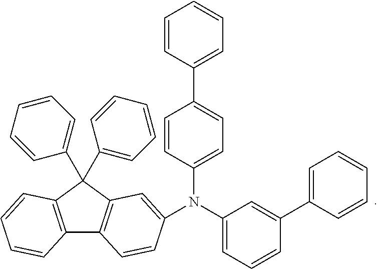 [SMILES: c1ccc(-c2ccc(N(c3cccc(-c4ccccc4)c3)c3ccc4c(c3)C(c3ccccc3)(c3ccccc3)c3ccccc3-4)cc2)cc1]

10. The method for preparing the cover layer molecular structure according to claim 7, wherein
the cover layer molecular structure has a refractivity of 1.05 to 2.15 for an incident light having a wavelength of 450 nm; and
the cover layer molecular structure has a refractivity of 1.85 to 2.05 for an incident light having a wavelength of 530 nm.

11. The method for preparing the cover layer molecular structure according to claim 7, wherein
the cover layer molecular structure has an extinction coefficient of 40 k to 100 k for an incident light having a wavelength of 340 to 380 nm.

12. An organic light emitting diode (OLED) device, comprising an anode substrate, a hole injection layer disposed on the anode substrate, a hole transport layer disposed on the hole injection layer, a light-emitting layer disposed on the hole transport layer, a hole blocking layer disposed on the light-emitting layer, an electron transport layer disposed on the hole blocking layer, an electron injection layer disposed on the electron transport layer, a cathode substrate disposed on the electron injection layer, a cover layer disposed on the cathode substrate and an encapsulation film layer disposed on the cover layer, wherein
a cover layer molecular structure of the cover layer comprises a macromolecular structure formed by bonding a first central structure to a second central structure, wherein
the first central structure is one of 9,9-dimethyl-2-bromofluorene, 2-bromo-9,9′-spirobifluorene, and 2-bromo-9,9-diphenylfluorene; and
the second central structure is either of 3-p-tolyl-4-m-tolyl-diphenylamine and 4,4′-bis(3,5-xylyl)-diphenylamine.

13. The OLED device according to claim 12, wherein the 9,9-dimethyl-2-bromofluorene has a molecular structure of: 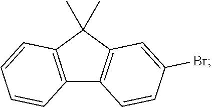 [SMILES: CC1(C)c2ccccc2-c2ccc(Br)cc21]
the 2-bromo-9,9′-spirobifluorene has a molecular structure of: 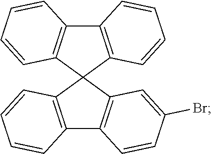 [SMILES: Brc1ccc2c(c1)C1(c3ccccc3-c3ccccc31)c1ccccc1-2]
the 2-bromo-9,9-diphenylfluorene has a molecular structure of: 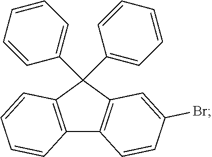 [SMILES: Brc1ccc2c(c1)C(c1ccccc1)(c1ccccc1)c1ccccc1-2]
the 3-p-tolyl-4-m-tolyl-diphenylamine has a molecular structure of: 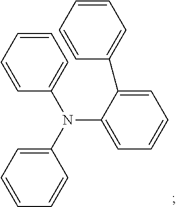 [SMILES: c1ccc(-c2ccccc2N(c2ccccc2)c2ccccc2)cc1]
and
the 4,4′-bis(3,5-xylyl)-diphenylamine has a molecular structure of:

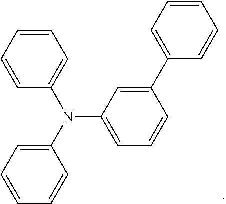 [SMILES: c1ccc(-c2cccc(N(c3ccccc3)c3ccccc3)c2)cc1]

14. The OLED device according to claim 12, wherein the molecular structure is:

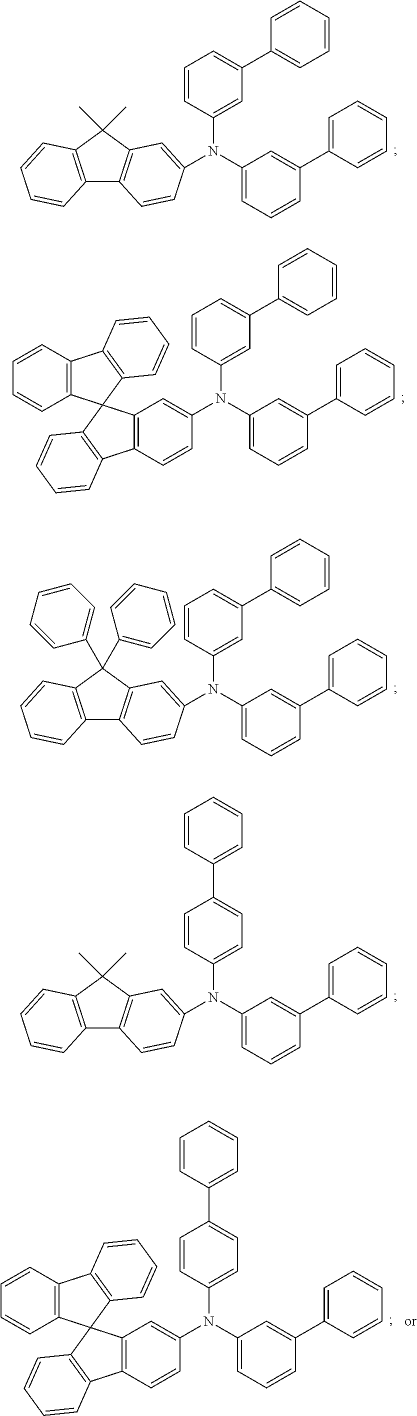 [SMILES: CC1(C)c2ccccc2-c2ccc(N(c3cccc(-c4ccccc4)c3)c3cccc(-c4ccccc4)c3)cc21 ; c1ccc(-c2cccc(N(c3cccc(-c4ccccc4)c3)c3ccc4c(c3)C3(c5ccccc5-c5ccccc53)c3ccccc3-4)c2)cc1 ; c1ccc(-c2cccc(N(c3cccc(-c4ccccc4)c3)c3ccc4c(c3)C(c3ccccc3)(c3ccccc3)c3ccccc3-4)c2)cc1 ; CC1(C)c2ccccc2-c2ccc(N(c3ccc(-c4ccccc4)cc3)c3cccc(-c4ccccc4)c3)cc21 ; c1ccc(-c2ccc(N(c3cccc(-c4ccccc4)c3)c3ccc4c(c3)C3(c5ccccc5-c5ccccc53)c3ccccc3-4)cc2)cc1]

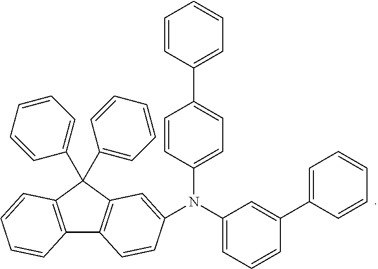 [SMILES: c1ccc(-c2ccc(N(c3cccc(-c4ccccc4)c3)c3ccc4c(c3)C(c3ccccc3)(c3ccccc3)c3ccccc3-4)cc2)cc1]

15. The OLED device according to claim 12, wherein the cover layer molecular structure is prepared by performing steps of:
reacting molecules of the first central structure and the second central structure for 48 hrs. in presence of palladium diacetate as a catalyst, tri-tert-butylphosphoniunm tetrafluoroborate as a ligand, and NaOt-Bu as a base in dewatered and deoxygenated toluene at 120° C., to produce the macromolecular structure.

16. The OLED device according to claim 12, wherein
the cover layer molecular structure has a refractivity of 1.05 to 2.15 for an incident light having a wavelength of 450 nm; and
the cover layer molecular structure has a refractivity of 1.85 to 2.05 for an incident light having a wavelength of 530 nm.

17. The OLED device according to claim 12, wherein the cover layer molecular structure has an extinction coefficient of 40 k to 100 k for an incident light having a wavelength of 340 to 380 nm.
